(19) United States
(12) Patent Application Publication
(10) Pub. No.: US 2010/0043803 A1
(43) Pub. Date: Feb. 25, 2010

(54) CONDOM HAVING TRANMISSION APPARATUS

(76) Inventor: Brian Osterberg, Petoskey, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/515,694

(22) PCT Filed: Nov. 20, 2007
(86) PCT No.: PCT/US07/85175

§ 371 Date: May 20, 2009

Related U.S. Application Data
(60) Provisional application No. 60/866,465, filed on Nov. 20, 2006.

(51) Int. Cl. A61F 6/04; A61N 1/00; A61F 5/00

(52) U.S. Cl. 128/844; 607/138; 607/143; 600/38

(57) ABSTRACT

A condom may include a device that may transmit a signal to a device remote from the condom. The remote device may be most any device capable of receiving a signal. For example, the remote device may be a light and/or sound generating and/or emitting device, ascent emitting device, or another device designed to stimulate the senses and enhance the experience of sexual intercourse. The signal device (i.e., the device that may transmit and/or receive a signal to/from a remote device) may be positioned at any position on or in the condom. For example, the signal device may be positioned in or on any part of the condom pouch, the condom frame, in the rolled up rim of the condom, and in or on any anchoring device for the condom. The device may be activated by a switch that may be triggered as a result of intercourse or sound.

### CROSS-REFERENCE TO RELATED APPLICATIONS

This Application is a Non-Provisional application of Provisional (35 USC 119(e)) application 60/866,465 filed on Nov. 20, 2006, the disclosure of which is incorporated herein by reference.

### FIELD OF THE INVENTION

The application describes a condom having an onboard transmitting and/or receiving apparatus that may operate to activate a device that is remote from the condom and/or may operate to receive a signal from such a device. More specifically, described herein is a male or female condom having positioned somewhere on the condom the means for transmitting or receiving a signal to or from a remote device. For example, the condom may operate to transmit a signal to control a device that is remote from the condom. Additionally, or alternatively, a remote device may operate to transmit a signal that is received by the condom and used to control a condom accessory.

### BACKGROUND OF THE INVENTION

It has been known in the art to mount devices on a male or female condom that may provide a direct physical stimulation to a user or a user's partner. However, it has not been known to construct a condom that has the ability to communicate with devices that are remote from the condom.

### SUMMARY OF THE INVENTION

A male or female condom may include a device that may transmit a signal to a device remote from the condom. The remote device may be most any device capable of receiving a signal. For example, the remote device may be a light and/or sound generating and/or emitting device, a scent emitting device, or another device designed to stimulate the senses and enhance the experience of sexual intercourse. The remote device may also be an apparatus that is worn by the user or the user's partner, such as a wristband, or jewelry/fashion accessory, a necklace, a ring, a bracelet, portable music player, cellular phone, BLUETOOTH enabled devices, etc.

The signal device (i.e., the device that may transmit and/or receive a signal to/from a remote device) may be positioned at any position on or in the condom. For example, the signal device may be positioned in or on condom, including any part of the condom pouch, the condom frame, in the rolled up rim of the condom, in or on any anchoring device for the condom (e.g., a sponge of a female condom), or between or on layers/bi-layers of the pouch. The signal device may be activated by any manner of switch that may be triggered as a result of intercourse or sound. By way of example, the switch may include a toggle switch, a membrane switch, a pressure sensitive switch, timer switch, a thermal switch, a fluid activated switch, or a microphone in electrical communication with the signal device such that a loud noise or a predetermined (programmed) pattern of sound may activate the signal device (e.g., THE CLAPPER).

In operation, the signal device may be activated as a result of an erect penis contacting or otherwise causing sufficient pressure to trip the switch. Alternatively, where the switch is a thermal switch, a switch may be preset to open upon reaching a temperature that is at or greater than the normal human body temperature of 98.6 Fahrenheit. Once activated, the signal device may emit a signal that may be received by the remote device. Upon receipt of the signal, the remote device may then perform a predetermined operation. For example, if the remote device is a stereo, upon receipt of the signal transmitted by the signal device, the stereo may commence playing a compact disc recording by the late Barry White.

In another embodiment, the condom assembly may also include a condom accessory that may be positioned anywhere on the condom including on or in the condom wall, on or in the anchor, positioned in a pocket or space defined in the wall or positioned on the frame of the condom or any part of the harness, etc. The condom accessory may include, for example, a vibration (or oscillation) device, a light and/or sound device, a lubricating or a scented fluid/gel holder/dispenser, a pillow, a solid device, or a pad and any combination thereof. One or more than one condom accessory may be used on the condom, depending on the model and/or the needs of the user. The condom accessories may be constructed to be deposable or reusable. Furthermore, the condom may be constructed so that the user may interchange the condom accessories to meet his or her needs.

The signal device may be in direct or remote electrical communication, wireless or audio/sound communication with one or more the condom accessories so as to control and/or activate the condom accessory. For example, where the condom accessory is a vibration device, a remote device (i.e., a key fob style transmitter, or remote control device, or any other device discussed above having the suitable electronic means positioned thereon) may be used to transmit a signal that is received by the signal device and results in the activation of the vibration device. Alternatively, it will also be appreciated that a condom mounted (i.e., on board) signal device may operable to activate condom mounted (i.e., on board) condom accessory by means of direct (wired) or remote (wireless) communication between the signal device and the condom accessory.

### BRIEF DESCRIPTION OF THE DRAWINGS

A better understanding of the condom disclosed herein may be had upon reference to the attached drawings, wherein like reference numerals refer to like parts throughout, and wherein:

FIG. 1 is a diagrammatic view of a signal device for use in connection with the condom;

FIG. 2 is a view of a male condom in communication with a remote device;

FIG. 3 is a perspective view of a female condom;

FIG. 4 is a partial view a female condom showing, specifically, the open end of the female condom.

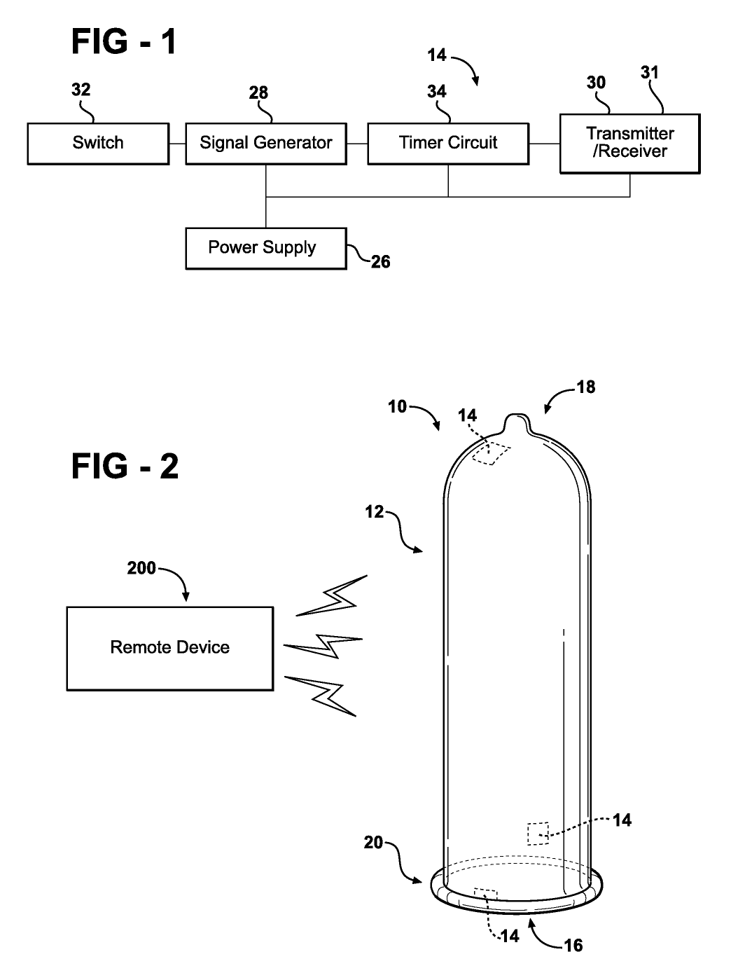

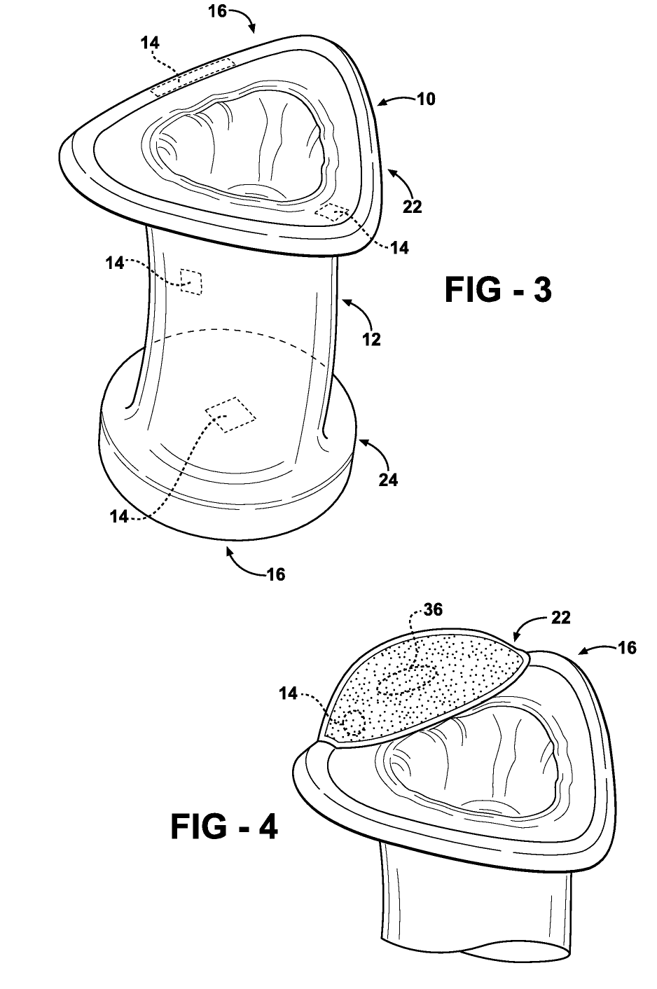

### DETAILED DESCRIPTION OF THE PREFERRED EMBODIMENTS

A condom may include a pouch and a signal device positioned on or within any part of the pouch. The condom may be a male or female condom having an open end and a closed end. The open end of the condom may include a rolled end of the pouch or a rigid or semi-rigid frame. In a female condom, an anchoring device (such as a sponge or other device known in the art) may be positioned within the pouch proximate the closed end. A signal device may include a power supply, a signal generator, such as an integrated circuit or microcontroller for generating a signal, a transmitter for transmitting the signal, a receiver for receiving a signal and a switch for activating the signal device apparatus. The signal device may also include a timer circuit to further control the operation of the signal device. A remote device that may receive a signal from the signal device may be positioned a distance from the pouch and may operate to receive and respond to the signal.

A condom 10 may include a pouch 12 and a signal device 14 mounted to any part of the pouch 12 or any device associated with the pouch 12. The condom 10 may be a male “M” or female “F” condom having an open end 16 and a closed end 18. The open end 16 of the condom 10 may include a rolled rim 20 of the pouch 12 or a rigid or semi-rigid frame 22. In a female condom “F”, an anchoring device 24 (such as a sponge or other device known in the art) may be positioned within the pouch 12 proximate the closed end 18. The signal device 14 may include a power supply 26, a signal generator 28, such as an integrated circuit or microcontroller, for generating a signal, a transmitter 30 for transmitting the signal, a receiver 31 and a switch 32 for activating the transmitting apparatus 14. The signal device 14 may also include a timer or clock circuit 34 to further control the operation of the signal device 14. Further, remote device 200 that may receive a signal from the signal device 14 may be positioned a distance from the pouch 12 of the condom 10 and may operate to receive and respond to the transmitted signal.

The signal device 14 may, for example, have construction similar to a modern electronic key fob and/or garage door opener. As such, the power supply 26 of the signal device 14 may include a lithium cell battery or other power device known in the art. Further, the signal generator 28 may include an integrated circuit or microcontroller (Dallas Semiconductors).

The transmitter 30, which may be in electrical communication with the signal generator, should be small in size, but may otherwise be constructed of commercially available hardware. For example, the transmitter may be constructed using the MAX1472 VHF/UHF PLL-based ASK-transmitter from Maxim or the AM-TX1-4xx AM transmitter module from ABACOM Technologies. Alternatively, the signal device 14 may simply include a transceiver chip (see e.g., the AMI Semiconductor AMIS-53000 or the Dallas Semiconductor DS276 Low-Power Serial Communications Transceiver), to manage the task of sending or receiving a signal.

The switch 32 may communicate with the signal generator 28 as the means for activating the signal generator 28. As mentioned above, the switch 32 may, for example, be a toggle switch, membrane switch, a thermal switch, timer switch, or a sound activated switch/microphone in electrical communication with the signal device such that a loud noise or a predetermined (programmed) pattern of sound may activate the signal device (e.g., THE CLAPPER).

Finally, a timer or clock circuit 34 may be incorporated into the signal device 14 as a means for controlling the emission or reception of a signal. Specifically, the timer circuit 34 may be used to control the duration of a signal transmission or to prevent the transmission of a further signal until the passage of a predetermined period of time.

The pouch 12 of the condom 10 may be constructed using synthetic materials such as polymeric materials in a manner similar to male and female condoms of known design in the art. However, natural materials such as latex or sheep intestine may also be used to construct the pouch 12. For example, the pouch 12 of a male design may be constructed in a manner similar to that of commercially available latex models of INSPIRAL® brand condom and a pouch of a female design may be constructed in a manner similar to commercially available latex VA® condom. Further, it will be appreciated that other future male and female pouch 12 designs could also be adapted to use a signal device 14.

As mentioned above, the signal device 14 may be positioned at any position on or within the pouch 12 of the condom 10. For example, the signal device 14 may be integrally molded into the pouch 12. Alternatively, the signal device 14 may be positioned within a pocket that is defined in the pouch 12. Further, where the condom 10 includes a pouch 12 for a female condom design, the signal device 14 may also positioned on or in the anchoring device or it may be positioned on or in the frame (including a pocket or chamber defined within the frame).

The remote device 200 may be positioned a distance from the condom 10 and operate to receive and respond to the signal generated by the signal generator 28 and emitted by the transmitter 30. As such, the remote device 200 may include a receiver that is tuned to receive the signal from the signal device 14. As mentioned above, the remote device 200 may be any manner of device that the user of the condom 10 may be interested in controlling. For example, the remote device may be a light and/or sound generating and/or emitting device (e.g., a lamp(s), a stereo, a radio, a strobe light, etc.), a scent emitting device (e.g., an automatic air freshener), or another device designed to stimulate the senses and enhance the experience of sexual intercourse. The remote device may also be an apparatus that is worn by the user or the user's partner, such as a wristband or a jewelry/fashion accessory, a necklace, a ring, a bracelet, a portable music player, a cellular phone, or Bluetooth enabled device, etc.

In operation, regardless of where the signal device 14 is positioned relative to the pouch 12 of the condom 10, the switch 32 of the signal device 14 may be arranged such that it is triggered during sexual intercourse as a result of the force, pressure and/or additional thermal energy generated by the action of an erect penis of the user, or body contact of the user or the user's partner. For example, if the signal device 14 is positioned in the anchoring device 24 (or in a pouch(es) defined in the wall) of a female condom design, a force resulting from manual stimulation or an erect penis impacting the anchoring device 24 (or the pouches) may result in the triggering of the switch 32. Alternatively, if the signal device 14 is positioned in an outer frame 22 or a pocket formed in a wall of the pouch 12 or the outer rim of a male condom design, the force/pressure generated by the installation of the condom 10 over the penis and/or the act of intercourse (e.g., force or pressure from body contact) may result in the triggering of the switch 32.

The triggering of the switch 32 may result in the generation of a signal by the signal generator 28 and the transmission of that signal by the transmitter 30. Further, as mentioned above, the timer or clock circuit 34 may be used to prevent the transmission of unnecessary or unwanted signals. For example, assuming that the remote device 200 is a massaging device that is worn by a user or the user's partner, the first signal transmitted by the condom 10 may be an ON signal for the massage device. However, to prevent the immediate subsequent transmission of an OFF signal, the timer circuit 34 may be programmed to prevent the transmission of a second signal for a predetermined period of time (e.g., 30 minutes).

In another embodiment, the condom 10 may include at least one condom accessory 36, for stimulating at least one of the five senses, and the signal device 14 may be in electrical communication with the condom accessories 36 so as to control and/or activate the condom accessory 36.

More specifically, the condom 10 include a condom accessory 36 that may be positioned on or in the condom wall, in a sponge (or other material that functions as a filler or cushion) in the condom, on or in an anchor for the condom (female), positioned in a pocket or space defined in the wall or positioned on the frame (female) of the condom or any part of a harness for the condom. The condom accessory 36 may include, for example, a vibration (or oscillation) device, a light and/or sound device, a lubricating fluid/gel or scented fluid/gel holder/dispenser, a pillow, a solid device, or a pad and any combination thereof. One or more than one condom accessory 36 may be used on the condom, depending on the model and/or the needs of the user. The condom accessories 36 may be constructed to be deposable or reusable. Furthermore, the condom may be constructed so that the user may interchange the condom accessories to meet his or her needs.

In operation, the signal device 14 may be in electrical or audio/sound communication with one or more the condom accessories 36 so as to control and/or activate the condom accessory 36. For example, where the condom accessory is a vibration device, a remote device 200 (i.e., a key fob style transmitter, or remote control device, or other devices identified above, such as wristband, jewelry, etc., having the necessary electronics) may be used to transmit a signal that is received by the signal device that results in the activation of the vibration device. Alternatively, it will also be appreciated that a condom mounted (i.e., on board) signal device may operable to activate condom mounted (i.e., on board) condom accessory by means of direct (wired) or remote (wireless) communication between the signal device and the condom accessory.

Therefore, having thus described the condom with transmitting apparatus, I claim:

1. A condom for transmitting a signal to a remote device comprising:
a condom having a pouch, the pouch having an open end and a closed end; and
a signal device positioned on the condom operable to transmit or receive a signal to or from a device remote from the condom.

2. The condom of claim 1, wherein the signal device comprises a signal generator, a transmitter in communication with the signal generator, and a switch in communication signal generator.

3. The condom of claim 2, wherein the switch comprises a thermal switch.

4. The condom of claim 2, wherein the switch is selected from a group consisting of a toggle switch, a fluid activated switch, a membrane switch, a pressure sensitive switch, a timer switch, and a sound activated switch.

5. The condom of claim 1, wherein the signal device further comprises a timer circuit.

6. The condom of claim 1, wherein the signal device further comprises a programmable circuit.

7. The condom of claim 1, further comprising a remote device operable to transmit or receive a signal from the signal device.

8. The condom of claim 1, further comprising a condom accessory.

9. The condom of claim 8, further comprising a remote device operable to transmit a signal to the condom to control the condom accessory.

10. The condom of claim 1, wherein the condom comprises a male condom.

11. The condom of claim 1, wherein the condom comprises a female condom.

12. The condom of claim 1, wherein the remote device is selected from a group consisting of a light emitting device, a sound emitting device and a scent emitting device.

13. The condom of claim 1, wherein the remote device comprises an apparatus operable to be worn by a person remote from the condom.

14. The condom of claim 1, where the apparatus operable to be worn by a person remote from the condom is selected from a group consisting of a wristband, a necklace, a ring, a bracelet, a portable music player, a cellular phone, and a Bluetooth enabled electronic device.

15. A condom for transmitting or receiving a signal in combination with a remote device that operates to receive or transmit a signal comprising:
a condom having a pouch, the pouch having an open end and a closed end;
a signal device positioned on the condom operating to transmit or receive a command signal to or from a device remote from the condom; and
an electronic device positioned a predetermined distance away from the condom and operating to transmit or receive a command signal to or from the signal device of the condom.

16. The condom of claim 15, wherein the condom comprises a male condom.

17. The condom of claim 15, wherein the condom comprises a female condom.

18. The condom of claim 15, wherein the electronic device comprises a signal generator, a transceiver in communication with the signal generator, and a switch in communication signal generator, the switch being selected from a group consisting of a toggle switch, a fluid activated switch, a membrane switch, a pressure sensitive switch, a timer switch, and a sound activated switch.

19. The condom of claim 15, further comprising a condom accessory positioned on the condom and operating to received a command signal from the electronic device.

20. The condom of claim 19, wherein the condom accessory is selected from a group consisting of a vibration device, an oscillation device, a light emitting device, a sound emitting device, and a fluid dispenser.
